(19) United States
(12) Patent Application Publication
(10) Pub. No.: US 2005/0036925 A1
(43) Pub. Date: Feb. 17, 2005

(54) Waste treatment apparatus

(76) Inventor: Mark Henry Butler, Barden Ridge (AU)

Correspondence Address: RYDER IP LAW, PC;DOUGLAS J. RYDER, 3669 CONCORD ROAD, DOYLESTOWN, PA, 18901, US

(21) Appl. No.: 10/497,998

(22) PCT Filed: Dec. 11, 2002
(86) PCT No.: PCT/AU02/01675

(30) Foreign Application Priority Data
Dec. 11, 2001 (AU) PR 9550

(51) Int. Cl. B02C 1/00; A61L 2/07

(52) U.S. Cl. 422/309; 422/26; 241/42; 241/606

(57) ABSTRACT

Waste treatment apparatus (200) a hopper (201) having an inlet for receiving a receptacle containing waste material and an outlet through which the receptacle is discharged to a first waste treatment chamber (202) having a first cutting mechanism (251) operative to shred the receptacle and to effect a first cutting treatment of the waste material contained in the receptacle, a second waste treatment chamber (203) having a second cutting mechanism (253) to effect a second cutting treatment of the shredded receptacle and waste material, a third waste treatment chamber (250) in communication with the second waste treatment chamber and a gate mechanism (206) at the outlet of the third waste treatment chamber (250) operative to permit discharge of the treated waste material from the apparatus; and means for introducing steam into the second treatment chamber (203) and the third treatment chamber (250).

### FIELD OF THE INVENTION

The present invention relates to waste treatment apparatus such as waste treatment apparatus for use in the sterilisation of infectious or quarantined waste. However, it will be appreciated that the invention is not limited to those particular uses and will find application in treating other types of waste which require sterilisation.

### BACKGROUND ART

Hospitals produce a large amount of infectious or quarantined waste. Generally, most hospitals have a number of infectious waste collection bins dispersed throughout the various wards and departments. These waste bins are periodically collected and removed to an off-site waste treatment facility for chemical sterilisation or high-temperature incineration of the waste, followed by sterilisation of the bin itself. This is an expensive process which suffers from many disadvantages.

One such disadvantage is the risk of environmental damage during the transport of infectious waste over public roads. In addition, the building and operation of an off-site chemical or incineration treatment centre represents a high capital cost outlay and requires expensive chemicals and/or fuels for operation.

### SUMMARY OF THE INVENTION

According to one aspect of the invention there is provided a waste treatment apparatus comprising:
(i) a hopper having an inlet for receiving a receptacle containing waste material and an outlet through which the receptacle is discharged;
(ii) a first waste treatment chamber having an inlet in communication with the outlet of the hopper and an outlet;
(iii) a first cutting mechanism operative to shred the receptacle and to effect a first cutting treatment of the waste material contained in the receptacle as the receptacle is discharged from the hopper;
(iv) a second waste treatment chamber having an inlet in communication with the outlet of the first waste treatment chamber and an outlet;
(v) a second cutting mechanism within the second waste chamber operative to effect a second cutting treatment of the shredded receptacle and waste material;
(vi) a third waste treatment chamber having an inlet in communication with the outlet of the second waste treatment chamber and an outlet;
(vii) a gate mechanism at the outlet of the second waste treatment chamber operative to permit discharge of the treated waste material from the apparatus;
(viii) means for introducing steam into the first treatment chamber at least whilst the first cutting mechanism is in operation; and
(ix) means for introducing steam into the second treatment chamber at least whilst waste material is transported from the inlet of the second treatment chamber to the outlet of the second treatment chamber.

### BRIEF DESCRIPTION OF THE DRAWINGS

FIG. 1 is a simplified, partially cross sectioned, side elevational view of a waste treatment apparatus according to one embodiment of the invention;

FIG. 2 is a cross sectional view of the shredding mechanism of the waste treatment apparatus shown in FIG. 1;

FIG. 3 is a partially cross sectioned side elevational view of the shredding mechanism shown in FIG. 2;

FIG. 4 is a rear elevation view of the waste treatment apparatus shown in FIG. 1;

FIG. 5 is a perspective view of the second cutting mechanism of the waste treatment apparatus shown in FIGS. 1 and 4;

FIG. 6 is a partially cross-sectioned top plan view of a waste treatment apparatus according to the second embodiment of the invention;

FIG. 7 is a partial cross-sectioned front elevational view of the waste treatment apparatus shown in FIG. 6;

FIG. 8 is a partially cross-sectioned side elevational view of the waste treatment apparatus shown in FIGS. 6 and 7;

FIG. 9 is an end view of the gate mechanism at the end of the discharge auger of the waste treatment apparatus shown in FIGS. 6 to 8;

FIG. 10 is a partially cross-sectional side elevational view of the gate mechanism shown in FIG. 9;

FIG. 11 is a view similar to FIG. 8 showing one half gate open;

FIG. 12 is a view similar to FIG. 11 with the second half gate and the first half gate partly open;

FIG. 13 is a view similar to FIG. 12 with the second half gate at the first gate half open;

FIG. 14 is a view similar to FIG. 13 with the second half gate and the first gate fully open; and

FIG. 15 is a cross-sectional view of a steam inlet valve for supplying steam to the second and third waste treatment chambers.

### MODES FOR CARRYING OUT THE INVENTION

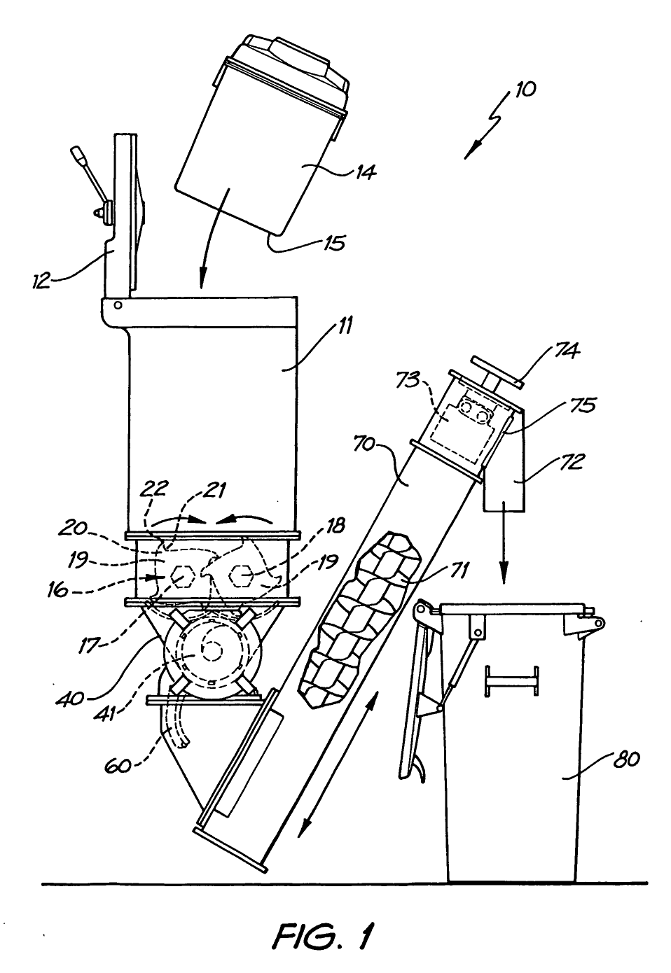

As shown in FIG. 1, a compact waste treatment apparatus 10 according to a first embodiment of the invention includes a hopper 11 which is sealed with respect to the environment by a reinforced door 12 of the type that might be used, for example, in an autoclave. The hopper is adapted to receive medical waste and particularly a sharps bin, for example, a B-D™ GUARDIAN™ nestable sharps collector or receptacle (model 22.71). The body of the hopper 11 is sized to receive the receptacle 14 snugly enough to prevent the container 14 from tumbling, twisting or turning within the hopper 11. This ensures that a lower portion 15 of the container 14 is caught and drawn into a first cutting and/or shredding mechanism 16 which is located in a first treatment at the outlet of the hopper 11.

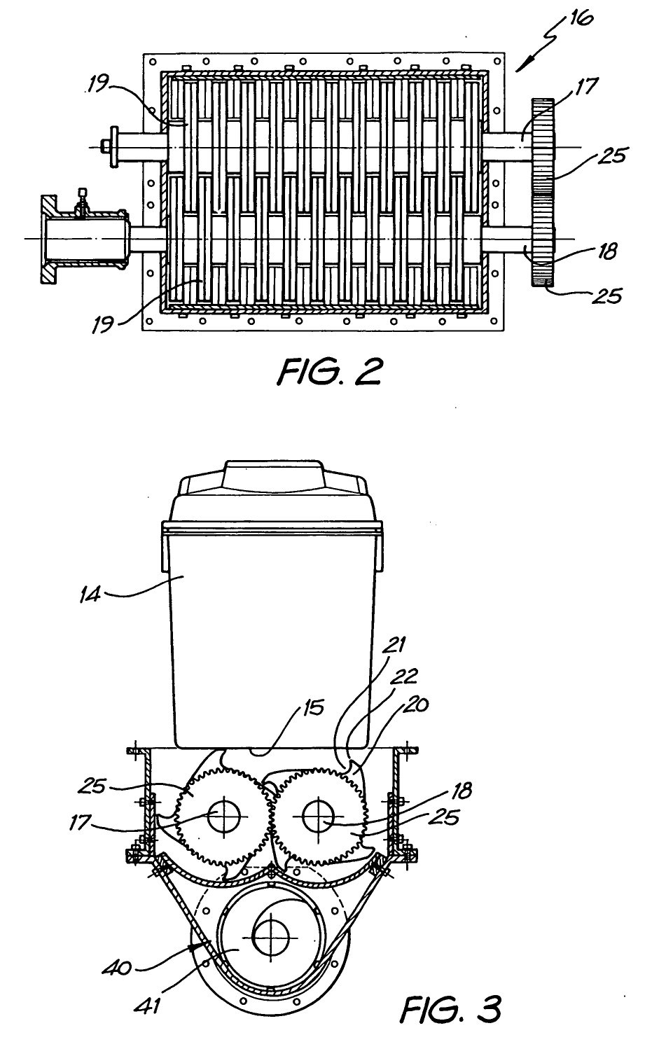

As shown in FIGS. 2 and 3, the cutting/shredder mechanism 16 comprises a pair of synchronised counter rotating shafts 17, 18 on which are rigidly mounted specially configured shredder blades 19. Each blade 19 comprises a number of equally spaced teeth 20, in this example, being 4 in number. The teeth are slightly concave 21 below the cutting edge 22. The blades arranged on a given shaft 17 or 18 are in alignment or registry. Because of the counter rotation of the shafts, the teeth converge, thereby gripping the waste or the container 14 and pulling it down and into the shredder 16. As shown in FIGS. 2 and 3, the two shafts 17 and 18 are synchronised with respect to one another by a pair of meshed synchronising gears 25, one mounted on each shaft.

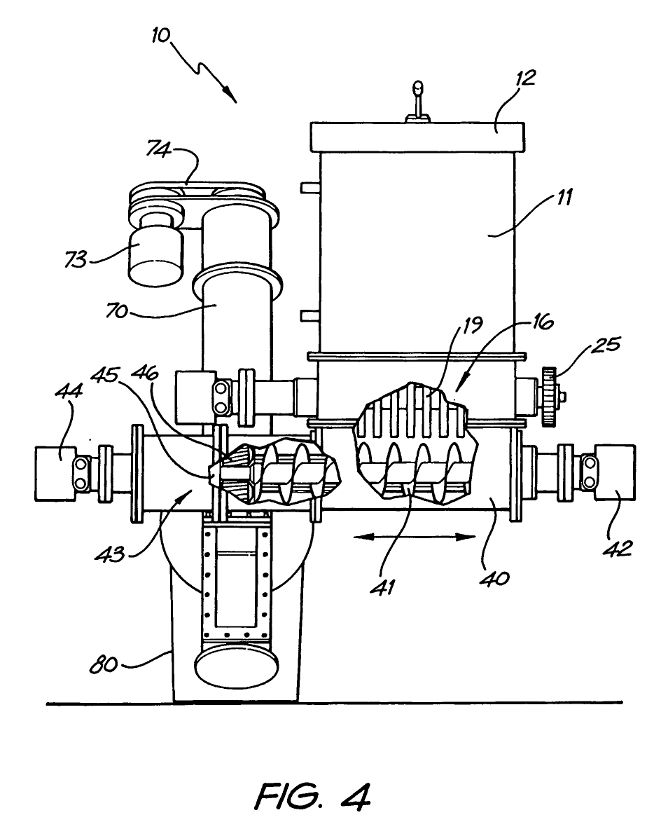

As shown in FIG. 4, the shredder mechanism 16 discharges its contents into the a second waste treatment chamber 40. The second chamber 40 contains a helical auger 41 which is driven from one end by a hydraulic motor 42. The auger 41 transports the waste, in a hot steam environment, toward the cutting head 43. The cutting head 43 comprises a hydraulic motor 44 which drives a planetary gearbox 45. A specially configured cutter 46 is mounted on each planet gear of the gearbox 45. Each cutter 46 comprises a generally cylindrical body 47 which terminates in a conical tip 48 (as shown in FIG. 5). The blades 49 of each cutter 46 are pitched and extend from the base 50 of each cutter to the conical tip 48.

The pitch of each blade 49 is anti-sense to conventional cutters. That is, each cutting blade 46 curves from the free end or tip 48 of the body 47 to the base of the body 42 adjacent to the planetary gear box 45 with the base end of the cutting blade 49 leading the free end of the cutting blade so that rotation of the cutter 46 drives the edge of the blades 49 into the waste material and urges the waste material in the second waste treatment chamber 40 away from the planetary gear box 45 rather than towards the planetary gear box 45.

Thus, the anti-sense pinch of the cutter blades 49 tends to reduce the accumulation of waste material around the cutting head 43. The space between the cutters 46 includes a bearing 51 for the leading end of the auger 41 in the second treatment chamber 40. As shown in FIG. 1 a transfer gate 60 separates the second waste treatment chamber 40 from the third waste treatment chamber 70.

Waste entering the third treatment chamber 70 is treated with steam and transported by auger 71 toward a discharge chute 72. The auger 71 is driven by a hydraulic motor 73 which in turn drives a chain 74 which rotates the third auger 71. A discharge gate separates the secondary treatment chamber 70 from the atmosphere. When the discharge gate 75 is open and the auger 71 is operated, waste is discharged into a bin 80.

Steam is introduced into the second treatment chamber 40 and the third treatment chamber 70 through self-cleaning steam valves 100 of the type depicted in FIG. 15. As shown, each steam valve 100 comprises a body 101 having a steam inlet 110. A reciprocating plunger 111 resides in the body 100 and is urged towards the steam outlet 112 by a compression spring 113. The degree of compression in the spring 113 is determined by the position of an adjustable back stop 114. The outer periphery 115 of the back stop 114 is threaded and these threads co-operate with internal threads 116 formed in the internal bore of the body 110. When the pressure of the steam entering the body 100 through the inlet 110 reaches a predetermined level, the plunger 111 is lifted against the spring bias. When the plunger 111 is lifted, the plunger tip 117 rises enough to allow steam to exit the outlet 112. When the steam pressure is relieved, the plunger 111 returns to its original position and the tip of the plunger 117 purges the outlet 112 of any obstructions or debris. The self cleaning valves 100 are distributed along the length and around the periphery of the second treatment chamber 40 and, around the third treatment chamber 70.

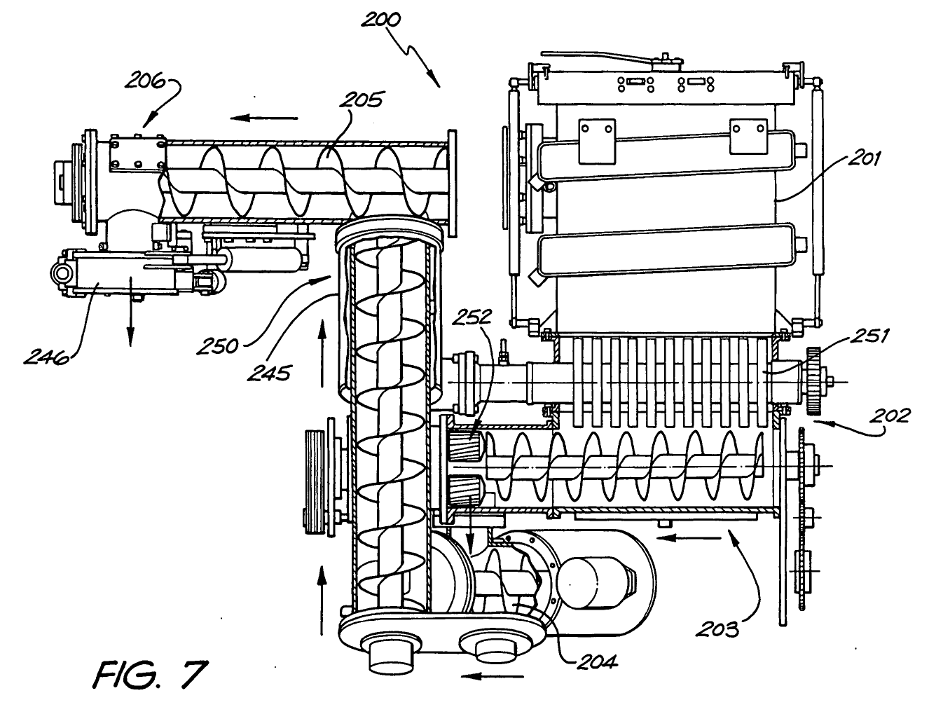

As shown in FIG. 9, the outlet gate mechanism 160 comprises an inner gate 161 and an outer gate 162. The inner gate 161 consists of a solid steel disk or plate 163 which is pivotally connected to the second waste treatment chamber 51. The connection between the plate 163 and the chamber 51 comprises a hinge 164 located above the second auger 52. The bottom edge of the disk 63 carries a reversed “J” shaped section 165. The section 165 engages the bottom of a terminal flange 166 of the second treatment chamber 51. This provides a positive mechanical engagement between the inner gate 161 and the chamber 51 when the inner gate 61 is closed.

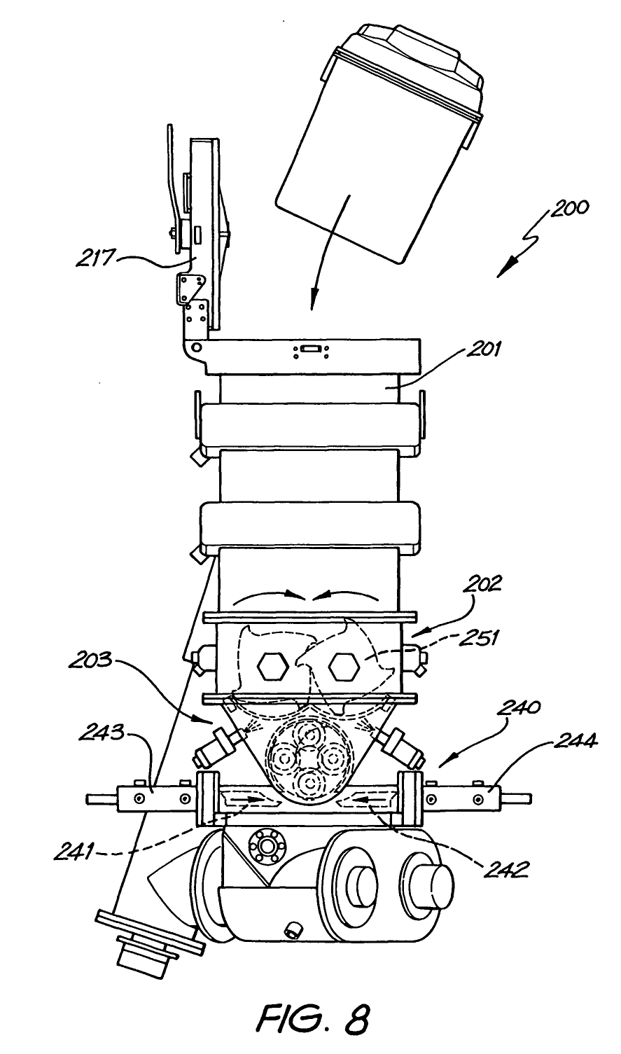

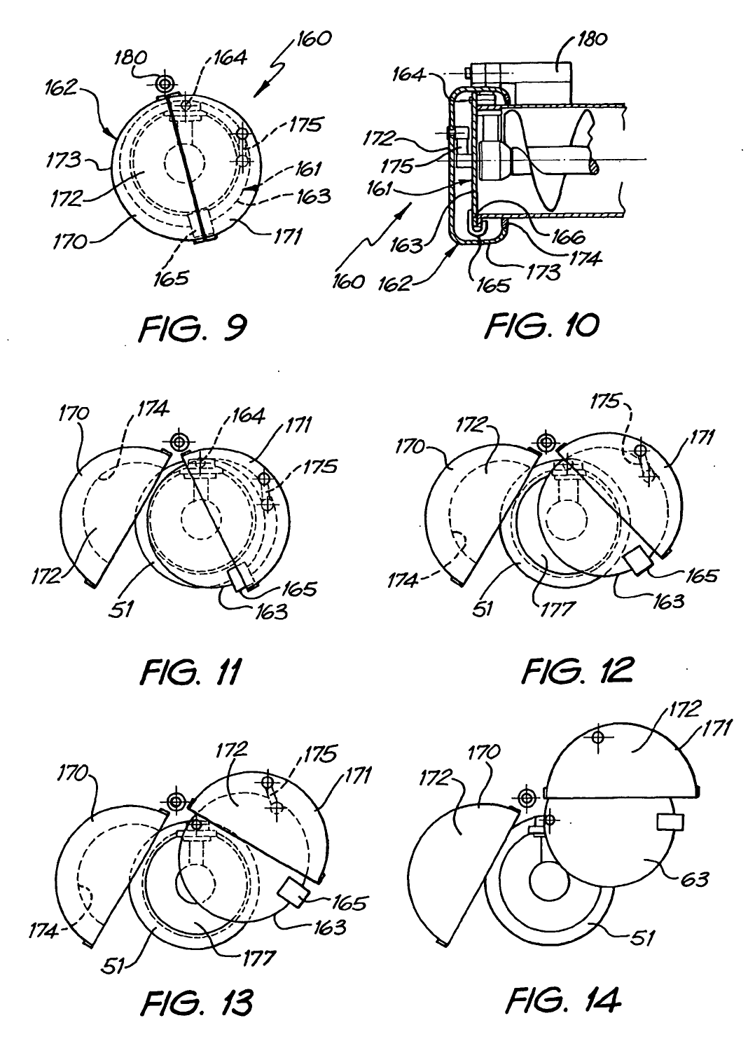

The opening and closing movement of the inner gate 161 is determined by the action of the outer gate 162. The outer gate 162 forms an outer covering and seal and as shown in FIG. 9 comprises two similar and co-operating half gates 170 and 171. Each half gate 170 and 171 further comprises a semi-circular front portion 172 and a semi-cylindrical side wall 173 (see FIG. 10) and a semi-circular sealing surface 174. Each sealing edge 174 is terminated with a section of “O” ring material to effect a seal between the outer gate 162 and the chamber 51. Similarly, the edges of the half gates 170 and 171 which meet together are also formed with grooves in which “O” ring sections are placed to effect a seal between the two half gates 170 and 171.

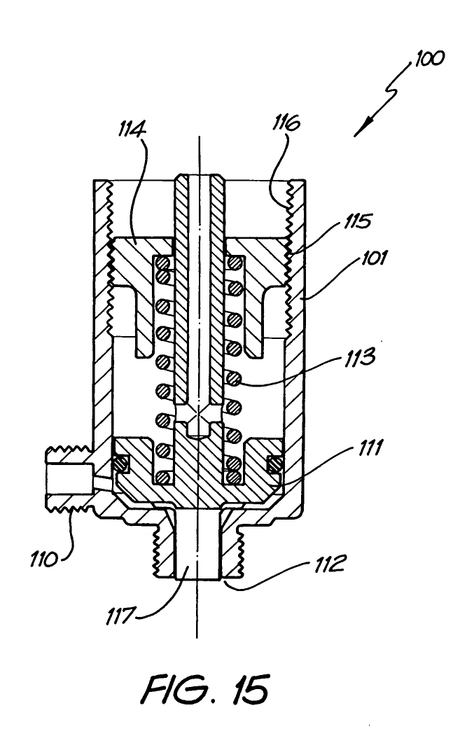

FIGS. 11 to 14 illustrate the sequence in which the inner gate 161 and the outer gate 162 are opened under the influence of a hydraulic actuator 180 mounted on the exterior of the second treatment chamber 51. In FIG. 9, the two half gates 170 and 171 are fully closed and held together to effect a seal between the two half gates 170 and 171 and between the outer gate 162 and the second treatment chamber 51. FIG. 11 illustrates the outer gate 162 as partially open. In this position, the inner gate 161 has been displaced slightly owing to the linkage 175 connected between the inner and outer gates 161 and 162. As shown in FIGS. 9 to 14, the link 175 extends between the outside surface of the inner gate 161 and the interior surface of the half gate 171. In this way, when the half gate 171 is opened, it lifts the inner gate 161 away from the opening 177 at the end of the second treatment chamber 51.

The inner gate 161 serves primarily as a mechanical barrier to the exit of material and also serves to wipe obstructions away from the opening 177 when the gates 161 and 162 are closed. The outer gate 162 serves the function of providing an effective steam seal between the interior of the second treatment chamber 51 and the environment.

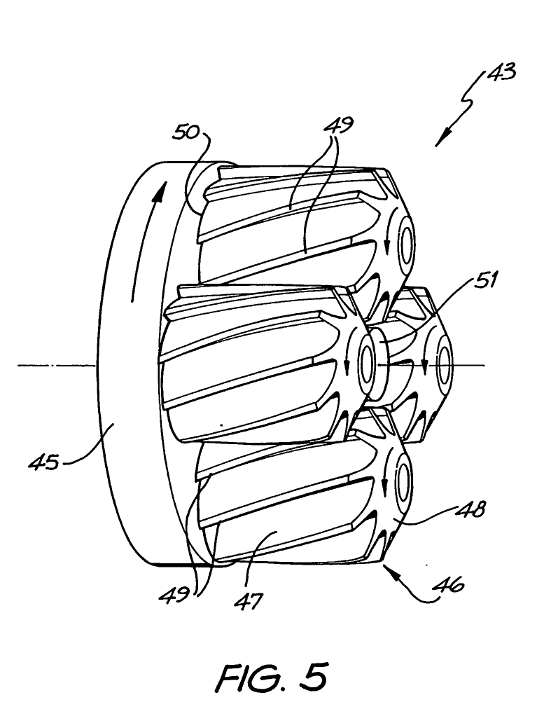

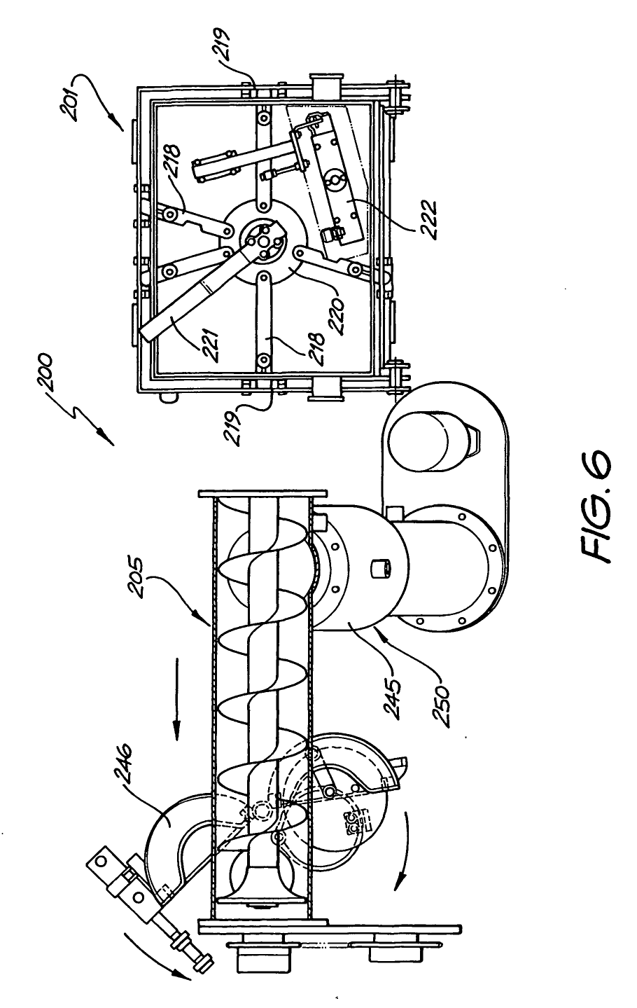

The second embodiment of the invention shown in FIGS. 6 to 8 is somewhat similar to the first embodiment in that the waste treatment apparatus 200 includes a loading hopper 201, a first treatment chamber 202 and a second treatment chamber 203 and a third treatment chamber 250. Steam is introduced into the second and third chambers 203 and 250 by valve 100 which are as described with respect to the first embodiment of the invention. In addition, this embodiment of the invention includes a transfer auger 204 between the second treatment chamber 203 and the third treatment chamber 250 as well as a discharge auger 205 leading from the third treatment chamber 250 to a discharge gate mechanism 206.

In the first chamber 202 there is a first cutting or shredding mechanism 251 and in the second chamber 203 there is a second cutting mechanism.

In this embodiment, the containment door 217 is locked closed by a plurality of latches 218 that engage catches 219 mounted on the door 217. The latches 218 are moved into and out of engagement with the catches 219 by a circular plate 220 operated by a lever 221. In the corner portion of the door 217 there is locking mechanism 222.

The second treatment chamber 203 has a discharge door 240 which consists of opposed half doors 241 and 242 operated by rams 243 and 244 respectively. The opposing portions of the half doors 241 and 242 are shaped to conform with the shape of the bottom of the first treatment chamber 202.

At the top of the third treatment chamber 250 there is a steam jacket 245 for maintaining the desired temperature of the waste material as it is transported towards the discharge conveyor 205. At the end of the discharge conveyor 205 there is a gate mechanism 246 similar to that shown in the first embodiment.

1. Waste treatment apparatus comprising:
(i) a hopper having an inlet for receiving a receptacle containing waste material and an outlet through which the receptacle is discharged;
(ii) a first waste treatment chamber having an inlet in communication with the outlet of the hopper and an outlet;
(iii) a first cutting mechanism operative to shred the receptacle and to treatment of the waste material contained in the receptacle as the receptacle is discharged from the hopper;
(iv) a second waste treatment chamber having an inlet in communication with the outlet of the first waste treatment chamber, and an outlet;
(v) a second cutting mechanism within the second waste chamber operative to effect a second cutting treatment of the shredded receptacle and waste material;
(vi) a third waste treatment chamber having an inlet in communication with the outlet of the second waste treatment chamber and an outlet;
(vii) a gate mechanism at the outlet of the third waste treatment chamber operative to permit discharge of the treated waste material from the apparatus;
(viii) means for introducing steam into the second treatment chamber at least whilst the second cutting mechanism is in operation; and
(ix) means for introducing steam into the third treatment chamber at least whilst waste material is transported from the inlet of the second treatment chamber to the outlet of the second treatment chamber.

2. Apparatus according to claim 1 wherein the hopper has an open top which is closed by a containment door shaped to receive the waste receptacle which is of a pre-determined shape and to deliver the receptacle to the outlet without twisting, turning or tumbling and wherein the outlet is at the bottom of the hopper.

3. Apparatus according to claim 1 and including an auger within the second waste treatment chamber.

4. Apparatus according to claim 3 wherein the second cutting means is at the end of the second waste treatment chamber adjacent to the outlet and including drive means for rotating the auger in a direction to feed the waste material from the inlet of the second waste treatment chamber towards the second cutting mechanism.

5. Apparatus according to claim 3 wherein the second cutting mechanism includes a planetary gear box having a plurality of planet gears, a stub axle projecting out of the gear box from each planet gear, and a generally cylindrical cutting head on each stub axle.

6. Apparatus according to claim 5 wherein each cutting head includes a body portion having a base adjacent the gear box and a free end face remote from the gear box, and a plurality of cutting blades spaced around the periphery of the body portion.

7. Apparatus according to claim 6 wherein each cutting blade curves from the free end of the cutting head to the base end of the cutting head with the base end of the cutting blade leading the free end of the cutting blade when the cutting head is rotated.

8. Apparatus according to claim 7 wherein the free end of the cutting head has a conical surface.

9. Apparatus according to claim 5 wherein the stub axles are equally radially spaced from the centre of the planetary gear box and extending from the centre of the planetary gear box there is a bearing for the leading end of the auger in the second waste treatment chamber.

10. Apparatus according to claim 3 wherein the auger is driven by an hydraulic motor mounted on the exterior of the second waste treatment chamber.

11. Apparatus according to claim 1 wherein the means for introducing steam into the second treatment chamber comprises a plurality of steam inlet valves mounted around and along the second treatment chamber.

12. Apparatus according to claim 11 and including a plurality of steam inlet valves mounted around and along the third treatment chamber.

13. Apparatus according to claim 2 wherein the containment door is firmly clamped to the hopper by a plurality of latches which are operated by a lever to engage catches on the hopper.

14. Apparatus according to claim 11 the second treatment chamber is circular in cross-section and wherein the outlet from the second treatment chamber is closed by a pair of opposed half doors slidably mounted on the second treatment chamber adjacent to its outlet and which are slidable between a first position in which the outlet is closed and a second position in which the outlet is open by an actuator.

15. Apparatus according to claim 14 wherein the inner face of each half door is arcuately shaped to conform to the shape of the interior of the second waste treatment chamber.

16. Apparatus according to claim 1 wherein the third waste treatment chamber is circular in cross section and includes an auger within the chamber.

17. Apparatus according to claim 1 wherein the gate mechanism includes an inner gate and an outer gate and wherein the outer gate is opened and closed by an actuator and the inner gate is connected to the outer gate so that it is opened and closed by the movement of the outer gate.

18. Apparatus according to claim 17 wherein the inner gate is a circular solid plate hinged to the second treatment chamber and connected to the outer gate by a linkage.

19. Apparatus according to claim 17 wherein the outer gate consists of two similar and co-operating half gates, with each half gate comprising a straight edge facing the other half gate, a semi-circular front portion, a semi cylindrical side wall extending around the outer periphery of the semi-circular front portion and an inwardly directing sealing surface around the side wall adapted to seal against the outside of the second treatment chamber.

20. Apparatus according to claim 19 including seals between the straight portions of each half gate and between the sealing surfaces of the half gates and the second waste treatment chamber.

21. Apparatus according to claim 1 and further including a transfer auger between the second treatment chamber and the third treatment chamber.

22. Apparatus according to claim 1 and further including a discharge auger leading from the third treatment chamber to the discharge gate mechanism.

23. Apparatus according to claim 1 wherein the first cutting mechanism includes a pair of synchronised, counter rotating shafts on which are mounted shredder blades, the blades being arranged on each shaft so as to be in register with the blades on the other shaft.

24. Apparatus according to claim 23 wherein each blade has a plurality of equally spaced teeth.

25. Apparatus according to claim 24 wherein each tooth has a concave cutaway portion beneath its cutting edge.

26. Apparatus according to claim 24 wherein each blade has four teeth.

27. Apparatus according to claim 23 wherein the shafts are contra rotated with the top portion of the blades moving towards one another so as to draw the receptacle into the first cutting mechanism.
